(19) United States
(12) Patent Application Publication
(10) Pub. No.: US 2026/0034075 A1
(43) Pub. Date: Feb. 5, 2026

(54) CHOLINE CARBOXYLIC ACID BASED IONIC LIQUIDS AS ANTIMICROBIAL AGENTS

(71) Applicant: University of Mississippi, University, MS (US)

(72) Inventors: Claylee M. CHISM, University, MS (US); Eden E.L. TANNER, University, MS (US)

(21) Appl. No.: 19/100,598

(22) PCT Filed: Jul. 31, 2023
(86) PCT No.: PCT/US2023/071300

§ 371 Date: Feb. 3, 2025

Related U.S. Application Data
(60) Provisional application No. 63/370,003, filed on Aug. 1, 2022.

(51) Int. Cl. A61K 31/14; A61K 31/19; A61K 31/194; A61K 31/20; A61K 31/201; A61K 31/22; A61P 31/04

(52) U.S. Cl. CPC A61K 31/14; A61K 31/19; A61K 31/194; A61K 31/20; A61K 31/201; A61K 31/22; A61P 31/04

(57) ABSTRACT

In one aspect, the disclosure relates to ammonium carboxylic acid ionic liquids, methods of making the ionic liquids, pharmaceutical compositions comprising the same, and methods of treating both Gram-negative and Gram-positive bacterial infections using same. In one aspect, the ionic liquids are biocompatible with mammalian cells. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/370,003, filed on Aug. 1, 2022, which is incorporated herein by reference in its entirety.

### BACKGROUND

Antimicrobial resistance is one of the leading and most urgent global healthcare and economic concerns. Repetitive misapplication of antimicrobial agents, especially at sublethal doses, leads to mutated bacteria that are resistant to multiple commercially available antimicrobial agents. As antibiotics and similar treatments become less effective against these new generations of “superbugs,” or resistant microbes, there is an urgent need to develop new effective and biocompatible therapies.

Ionic liquids (ILs) are a class of compounds that are liquid below 100° C. and nonvolatile, consisting of bulky charged cations and anions rather than electrically neutral molecules. These salts possess beneficial properties including being extremely tunable, which means that bulk properties, including interactions with biomaterials or bacteria, can be manipulated through alteration of the chemical structure. This tunability has led to their emergence as promising tools to solve a range of biomedical problems including applications in drug delivery and protein stabilization.

Despite advances in ionic liquid research, there is still a scarcity of ionic liquids that are potent and efficacious against both Gram-positive and Gram-negative bacteria while also being biocompatible and inexpensive to synthesize in bulk quantities. These needs and other needs are satisfied by the present disclosure.

### SUMMARY

In accordance with the purpose(s) of the present disclosure, as embodied and broadly described herein, the disclosure, in one aspect, relates to ammonium carboxylic acid ionic liquids, methods of making the ionic liquids, pharmaceutical compositions comprising the same, and methods of treating both Gram-negative and Gram-positive bacterial infections using same. In one aspect, the ionic liquids are biocompatible with mammalian cells.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the cholinium carboxylate ionic liquid general structure.

FIG. 2 shows that minimum bactericidal concentration (MBC) values for E. coli are highly dependent on the identity and molar ratio of the anion of the ionic liquid (IL). Full names, structures, and characterizations listed in the examples.

FIGS. 3A-3F show anions with chains of 4-12 carbons show greatest antimicrobial efficacy with a peak at n=10. FIG. 3A: Saturated 1:1, FIG. 3B: 2-position unsaturated 1:1, FIG. 3C: 3-position unsaturated 1:1, FIG. 3D: saturated 1:2, FIG. 3E: 2-position unsaturated 1:2, and FIG. 3F: 3-position unsaturated 1:2. Trends in carbon chain lengths of 4-12 carbons with commercially available anions.

FIG. 4 shows ionic liquid treatment initiates interactions with bacterial membranes forming a coating around the cells. E. coli cells treated with no IL, sublethal dosage of four of the top four candidates (top row), and lethal dosage (bottom row) to show the change in morphology of the bacterial membranes. [Scale bar: 50 μm]

FIG. 5 shows interactions with ionic liquids elongate the lag phase even at sublethal concentrations as low as one-sixteenth of the lethal dose. OD₆₀₀ versus time for E. coli growth starting at the onset of the log phase to depict how interactions with IL even at concentrations well below that of the lethal concentration can affect inhibition.

FIGS. 6A-6C show altering ionic liquid concentration reduces the magnitude of the E. coli growth rate constant by >70%. FIG. 6A: Untreated, FIG. 6B: sublethal, and FIG. 6C: lethal data from FIG. 5 was fit using first-order kinetics to extrapolate the rate constants, keeping in mind that equations in FIGS. 6A-6B will have positive values while that in FIG. 6C will be negative.

FIG. 7 shows brightfield microscopy images convey minimal toxicity to HEK-293 cells until the MBC is doubled. When compared to the untreated cells (to the left), the first three concentrations of all four ILs show few to no toxic effects. However, once the MBC is doubled and quadrupled, the cells appear unhealthy and debris begins to appear in the field of view. [Scale bar: 100 μm]

FIGS. 8A-8D show ionic liquids show minimal toxicity below four times the MBC. FIG. 8A: Choline decanoate 1:1, FIG. 8B: choline decanoate 1:2, FIG. 8C: choline 2-decenoate 1:2, and FIG. 8D: choline 3-decenoate 1:2 was added to human embryonic kidney cells at one-fourth, one-half, equal to, two times, and four times the MBC and compared to untreated healthy cells to pinpoint at which concentration of IL the cells see decreased viability.

FIG. 9 shows a shift in optimal anion chain length is seen when changing from Gram negative to Gram positive bacteria. MBC values are at a minimum at n=10 for E. coli with a steady rise as carbons are added, while the lower limit for Methicillin-resistant Staphylococcus aureus (MRSA) is found at n=12. When focusing on the saturated chains, there is a steady increase from 10-12 for E. coli and a steady decrease from 10-12 for MRSA.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are ionic liquids including choline and an anion including the conjugate base of a substituted or unsubstituted C2-C20 linear or branched fatty acid. In one aspect, the fatty acid can be a saturated fatty acid, a monounsaturated fatty acid, a polyunsaturated fatty acid, or any combination thereof. In another aspect, the anion can be selected from butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof.

In some aspects, the ionic liquids are free from, or substantially free from, geranic acid and/or geranate anion. In another aspect, the molar ratio of the choline to the fatty acid is from about 1:1 to about 1:4, or about 1:1 to about 1:2, or is about 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, or about 1:4, or a combination of any of the foregoing values, or a range encompassing any of the foregoing values. In any of these aspects, the ionic liquid can be biocompatible and/or non-toxic.

In one aspect, the ionic liquid can be choline decanoate having a molar ratio of choline to decanoic acid of about 1:2, choline 3-decenoate having a molar ratio of choline to 3-decenoic acid of about 1:2, choline 2-decenoate having a molar ratio of choline to 2-decenoic acid of about 1:2, or any combination thereof.

In another aspect, the ionic liquids are inexpensive to prepare. In a still further aspect, the compounds used to prepare the ionic liquids do not require purification prior to ionic liquid synthesis, saving time and preparation steps. In still another aspect, the disclosed ionic liquids are more than 10 times more effective at killing bacteria than currently known ionic liquids. In yet another aspect, the ionic liquids retain effectiveness when diluted in aqueous solution and can be applied from aqueous solution.

In one aspect, disclosed herein are compositions including the ionic liquids. In one aspect, the compositions can be free from, or substantially free from, alcohols such as ethanol, isopropyl alcohol, or the like. In another aspect, the compositions can be free from, or substantially free from, pH adjusters, antimicrobial peptides, acrylate- and acrylamide-based polymers, dendrimers, nylon or nylon-type polymers, vinyl polymers, polycarbonates, polynorbornenes, guanide or biguanide polymers, polyurethanes, polystyrene polymers, polyvinylpyridine polymers, polyvinyl alcohol, skin conditioners, drying time enhancers, dyes, fragrances, gelling agents, humectants, emollients, fragrance agents, or the like. In an alternative aspect, the compositions can include one or more pharmaceutically acceptable carriers or excipients. In still another aspect, the ionic liquids or compositions including the ionic liquids can be applied topically, intranasally, intravenously, on surfaces, and/or in or around wounds or on bandages, gauze, and the like. In one aspect, the ionic liquid can be used to modify the surface of another material. In a further aspect, the material can be a polymeric substrate. In still another aspect, the polymeric substrate can comprise nanoparticles that are less than 1000 nm in size.

In one aspect, the ionic liquid can have a minimum bactericidal concentration (MBC) for E. coli of from about 0.5 mM to about 1750 mM, or from about 0.5 mM to about 100 mM, or of about 0.5, 1, 5, 10, 15, 20, 25, 30, 35, 40, 45, 50, 55, 60, 65, 70, 75, 80, 85, 90, 95, 100, 250, 500, 750, 1000, 1250, 1500, or about 1750 mM, or a combination of any of the foregoing values, or a range encompassing any of the foregoing values. In another aspect, the ionic liquid can have an MBC for methicillin-resistant Staphylococcus aureus (MRSA) of from about 0.5 mM to about 3 mM, from about 1 mM to about 2.25 mM, or of about 0.5, 0.75, 1, 1.25, 1.5, 1.75, 2, 2.25, 2.5, 2.75, or about 3 mM, or a combination of any of the foregoing values, or a range encompassing any of the foregoing values.

Also disclosed herein is a method for treating or preventing an infection caused by bacteria in a subject, the method including at least the step of administering a disclosed ionic liquid to the subject. In some aspects, the bacteria can be Gram-positive bacteria, Gram-negative bacteria, or both. In one aspect, the Gram-positive bacteria can be methicillin-resistant Staphylococcus aureus (MRSA), Mycobacterium tuberculosis, or both. In another aspect, the Gram-negative bacteria can be Escherichia coli.

In one aspect, the subject can be a mammal or a bird, including, but not limited to, a human, cat, dog, horse, cattle, sheep, goat, hamster, guinea pig, rabbit, mouse, rat, chicken, turkey, duck, goose, or parrot.

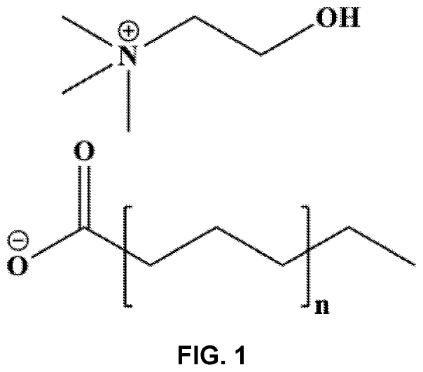

In one aspect, and without wishing to be bound by theory, ILs with quaternary ammonium cations (FIG. 1) may have substantial antimicrobial activity because of the anti-electrostatic properties of the hydrophilic head group. In a further aspect, choline is believed to have not only strong inhibitory effects against microbes but also minimal toxicity to mammalian cells when paired with biologically safe anions. In still another aspect, lipophilicity of both the cation and the anion of an IL may be important to the overall efficacy of the IL as bactericide against both Gram-positive and Gram-negative bacteria. While Gram-positive species lack an outer membrane, they are encompassed by a thick cell envelope consisting of numerous layers of peptidoglycan (30-100 nm). Meanwhile, Gram-negative bacteria are surrounded by a lipopolysaccharide outer membrane, but have only a thin peptidoglycan layer (2-10 nm) in the periplasmic space between the inner and outer membranes. In one aspect, and without wishing to be bound by theory, hydrophobic cations may insert themselves into the phospholipid bilayer of both Gram-negative and Gram-positive bacteria and causing disruption, which in turn contributes to cell death.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a Gram-negative bacterial species,” “a cation,” or “an anion,” include, but are not limited to, mixtures or combinations of two or more such Gram-negative bacterial species, cations, or anions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value.

Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less' and should be interpreted to include the specific ranges of ‘about x,’ ‘about y,’ and ‘about z’ as well as the ranges of ‘less than x,’ less than y,’ and ‘less than z.’ Likewise, the phrase ‘about x, y, z, or greater’ should be interpreted to include the specific ranges of ‘about x,‘ ’ about y,’ and ‘about z’ as well as the ranges of ‘greater than x,’ greater than y,’ and ‘greater than z.’ In addition, the phrase “about ‘x’ to ‘y,’” where ‘x’ and ‘y’ are numerical values, includes “about ‘x’ to about ‘y.’”

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an antimicrobial compound refers to an amount that is sufficient to achieve the desired improvement in the property modulated by the formulation component, e.g. achieving the desired level of bactericidal activity. The specific level in terms of wt % in a composition required as an effective amount will depend upon a variety of factors including the amount and type of bacterium being treated, identities of the cation and anion, surface or tissue being treated, and the like.

As used herein, the terms “optional” or “optionally” means that the subsequently described event or circumstance can or cannot occur, and that the description includes instances where said event or circumstance occurs and instances where it does not.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a bacterial infection. The effect can be therapeutic in terms of a partial or complete cure of a disease, condition, symptom, or adverse effect attributed to the disease, disorder, or condition. The term “treatment” as used herein can include any treatment of a bacterial infection in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

“Biocompatible” as used herein refers to a compound or composition, such as, for example, an ionic liquid, that does not damage or harm living tissue in a subject. In one aspect, a biocompatible material does not kill any living cells or trigger an immune response in a subject when the compound or composition is administered or applied to the subject. In one aspect, the ionic liquids disclosed herein are biocompatible.

As used herein, “microbicidal” refers to a compound or composition that kills microorganisms including bacteria and/or fungi, while “antimicrobial” kills microorganisms and/or stops their growth, and “bactericidal” compounds and compositions specifically kill bacteria but may or may not act against other types of microorganisms. In one aspect, the ionic liquids disclosed herein are microbicidal, antimicrobial, bactericidal, or a combination thereof.

“Minimum bactericidal concentration” or “MBC” as used herein refers to the lowest concentration of a bactericidal compound required to kill a given bacterium. Exemplary methods for determining MBC are provided in the Examples.

### Pharmaceutical Compositions

In one aspect, the ionic liquids or pharmaceutical compositions including the same can be applied topically to combat or prevent bacterial infections.

Unless otherwise specified, temperatures referred to herein are based on atmospheric pressure (i.e. one atmosphere).

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. An ionic liquid comprising choline and an anion comprising the conjugate base of a substituted or unsubstituted C2-C20 linear or branched fatty acid.

Aspect 2. The ionic liquid of aspect 1, wherein the fatty acid comprises a saturated fatty acid, a monounsaturated fatty acid, a polyunsaturated fatty acid, or any combination thereof.

Aspect 3. The ionic liquid of aspect 1 or 2, wherein the anion comprises butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof.

Aspect 4. The ionic liquid of any one of aspects 1-3, wherein a molar ratio of the choline to the anion is from about 1:1 to about 1:4.

Aspect 5. The ionic liquid of aspect 4, wherein a molar ratio of the choline to the anion is from about 1:1 to about 1:2.

Aspect 6. The ionic liquid of any one of aspects 1-5, wherein the ionic liquid comprises choline decanoate having a molar ratio of choline to decanoic acid of about 1:2, choline 3-decenoate having a molar ratio of choline to 3-decenoic acid of about 1:2, choline 2-decenoate having a molar ratio of choline to 2-decenoic acid of about 1:2, or any combination thereof.

Aspect 7. A composition comprising the ionic liquid of any one of aspects 1-6, wherein the composition is substantially free of alcohol.

Aspect 8. The composition of aspect 7, further comprising at least one pharmaceutically-acceptable carrier or excipient.

Aspect 9. The ionic liquid or composition of any one of aspects 1-8, wherein the ionic liquid or composition is biocompatible.

Aspect 10. The ionic liquid or composition of any one of aspects 1-9 wherein the ionic liquid or composition has a minimum bactericidal concentration (MBC) for E. coli of from about 0.5 mM to about 1750 mM.

Aspect 11. The ionic liquid or composition of any one of aspects 1-9, wherein the ionic liquid or composition has a minimum bactericidal concentration (MBC) for E. coli of from about 0.5 mM to about 100 mM.

Aspect 12. The ionic liquid or composition of any one of aspects 1-9, wherein the ionic liquid or composition has a minimum bactericidal concentration (MBC) for methicillin-resistant Staphylococcus aureus (MRSA) of from about 0.5 mM to about 3 mM.

Aspect 13. The ionic liquid or composition of any one of aspects 1-9, wherein the ionic liquid or composition has a minimum bactericidal concentration (MBC) for methicillin-resistant Staphylococcus aureus (MRSA) of from about 1 to about 2.25 mM.

Aspect 14. A method for treating or preventing an infection caused by bacteria in a subject, the method comprising administering the ionic liquid or composition of any one of aspects 1-13 to the subject.

Aspect 15. The method of aspect 14, wherein the subject is a mammal or bird.

Aspect 16. The method of aspect 15, wherein the mammal is a human, cat, dog, horse, cattle, sheep, goat, hamster, guinea pig, rabbit, mouse, or rat.

Aspect 17. The method of aspect 15, wherein the bird is a chicken, turkey, duck, goose, or parrot.

Aspect 18. The method of any one of aspects 14-17, wherein the ionic liquid or composition is administered topically.

Aspect 19. The method of any one of aspects 14-18, wherein the bacteria comprise Gram-positive bacteria, Gram-negative bacteria, or both.

Aspect 20. The method of aspect 19, wherein the Gram-positive bacteria comprise E. coli.

Aspect 21. The method of aspect 19, wherein the Gram-negative bacteria comprise methicillin-resistant Staphylococcus aureus (MRSA).

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

Materials: Choline bicarbonate (80% in water) and commercially available carboxylic acids were obtained through Sigma-Aldrich (St. Louis, MO, USA) and TCl America (Portland, OR, USA). Tryptone, yeast, agar, and sodium chloride for lysogeny broth were acquired from Fisher Scientific (Hampton, NH, USA). E. coli [BL21(DE3)] and MRSA [BAA-1708] were obtained from Thermo Scientific (Waltham, MA, USA) and ATCC (Manassas, VA, USA), respectively.

Synthesis and Characterization of ILs. Choline bicarbonate and carboxylic acids were combined in either 1:1 or 1:2 molar ratios via a salt metathesis reaction (Scheme 1) as described previously. Each IL was then dried using a rotary evaporator (15 mbar, 60° C. for 1 hr) and vacuum oven (60° C. for 48 hr). The water content was then obtained using a Karl Fischer coulometric titrator, and the IL was characterized using proton Nuclear Magnetic Resonance (NMR) spectroscopy. NMR characterization for ILs disclosed herein can be found in Example 2.

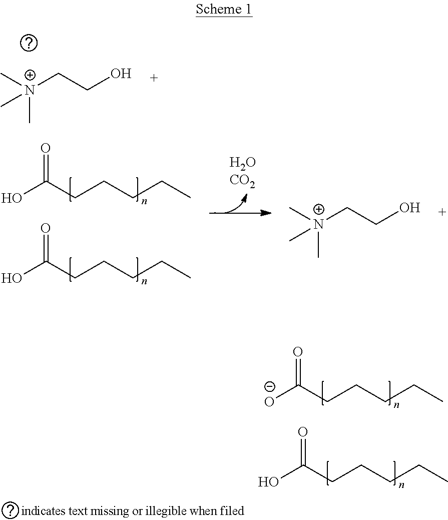

Scheme 1 shows representative ionic liquid synthesis. Salt metathesis reaction of choline bicarbonate and carboxylic acid in a 1:2 molar ratio to form cholinium carboxylate 1:2 illustrates the extraneous production of water and carbon dioxide.

Bacterial Cell Culturing. Expression cells from frozen stock were streaked onto a kanamycin-selected lysogeny broth (LBKan) agar plate using a sterile loop and incubated overnight at 37° C. One colony was then taken from the plate the next day to inoculate 50 mL of sterile LBKan broth and shaken at 37° C. until reaching a growth concentration of 4×10⁸ CFU/mL. This protocol was used for bacterial cell culture preparation in all of the experiments described below.

Minimal Bactericidal Concentration (MBC) Measurements. E. coli cells were cultured as described above and aliquoted into sterile tubes. 1%, 10%, and 100% (w/v) solutions of IL in sterile water (correcting for water percentage of the IL) were prepared and sonicated for 15 minutes at 40° C. before addition to the liquid culture. IL solutions were then added to E. coli at varying percentages (v/v), allowing calculation of the actual concentration of IL that was added to the culture. 100 μL of each solution was then spread onto an LBKan agar plate and incubated overnight at 37° C. MBC was determined as the lowest concentration of IL that resulted in no growth on the plate. Each IL solution was tested in triplicate.

Brightfield Microscopy Imaging. Untreated E. coli cells along with cells treated with lethal (MBC) and sublethal (one-half MBC) concentrations of four of the top candidates [choline decanoate 1:1 (CADA 1:1), choline decanoate 1:2 (CADA 1:2), choline 2-decenoate 1:2 (CA2DE 1:2), and choline 3-decenoate 1:2 (CA3DE 1:2)] were imaged with a Nikon Eclipse Ts2 inverted microscope with a 40× objective after growing overnight to view any topographical changes of the outer structures of the bacteria.

E. coli Kinetic Study. A 96-well plate was filled with 180 μL of E. coli cells that were cultured as mentioned above and treated with 20 μL CADA 1:1 of concentrations ranging from the lethal concentration to one-sixteenth of that concentration to determine how the growth inhibition is affected with changing concentration of the IL. 20 μL of growth media was added to the “untreated” cells such that all wells contained a total volume of 200 μL. The plate was heated to 37° C. with triplicate OD₆₀₀ measurements taken every 20 minutes over the course of 16 hours with a Biotek Synergy H1 microplate reader.

Cytotoxicity Studies. Human embryonic kidney (HEK-293) cells were treated in vitro with four of the top candidates (CADA 1:1, CADA 1:2, CA2DE 1:2, and CA3DE 1:2) to determine the concentration at which the IL dosage is toxic to human cells. 1% (w/v) solutions of each IL in sterile water were prepared similarly to the MBC experiments before addition at five different concentrations. The HEK cells were incubated with the IL treatments for 24 hours and then prepared with CellTiter-Glo(R) Luminescent Cell Viability reagent (100 μL per well). Brightfield microscopy images were used to qualitatively depict the concentration at which the cells were no longer viable, while a 96-well plate reader was used to quantitatively determine the percent viability of the cells at each concentration of the ILs. Quantitative measurements were performed in triplicate.

Growth Inhibition of Methicillin Resistant Staphylococcus Aureus (MRSA). 10% (w/v) solutions of IL in sterile water were prepared similarly to the E. coli MBC experiments. ILs with anion chain lengths of 8-12 carbons at 1:2 mole ratio of choline to anion were added to MRSA at varying concentrations to determine the lowest concentration in which growth was inhibited. Concentrations were then compared to values obtained for E. coli for each solution.

### Example 2: Characterization of Ionic Liquids and Anion Structures

Choline Butanoate 1:1 (CABA 1:1) ¹H NMR (400 MHz, d6-DMSO) δ 3.89-3.81 (m, 2H), 3.45 (t, J=5.0 Hz, 2H), 3.15 (s, 9H), 1.86 (t, J=7.0 Hz, 2H), 1.42 (h, J=7.4 Hz, 2H), 0.81 (t, J=7.3 Hz, 3H).

Choline Butanoate 1:2 (CABA 1:2) ¹H NMR (400 MHz, d6-DMSO) δ 3.84 (dq, J=5.4, 2.6 Hz, 2H), 3.42 (dq, J=4.7, 2.6 Hz, 2H), 3.12 (s, 9H), 2.04 (ddd, J=7.8, 4.9, 3.0 Hz, 4H), 1.46 (h, J=7.3 Hz, 4H), 0.84 (td, J=7.4, 1.7 Hz, 6H).

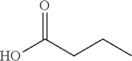

Choline 2-Butenoate 1:1 (CA2BE 1:1) ¹H NMR (400 MHz, d6-DMSO) δ 6.30 (dtd, J=13.7, 6.9, 3.0 Hz, 1H), 5.67 (dd, J=15.2, 2.1 Hz, 2H), 3.83 (q, J=4.1 Hz, 2H), 3.49-3.42 (m, 2H), 3.15 (s, 9H), 1.66 (d, J=7.0 Hz, 3H).

Choline 2-Butenoate 1:2 (CA2BE 1:2) ¹H NMR (400 MHz, d6-DMSO) δ 6.55 (ddq, J=16.1, 7.0, 2.9 Hz, 2H), 5.78 (dd, J=15.6, 2.3 Hz, 2H), 3.85 (dq, J=5.6, 2.7 Hz, 2H), 3.44 (tt, J=5.1, 2.6 Hz, 2H), 3.14 (d, J=1.8 Hz, 9H), 1.74 (dd, J=6.9, 1.9 Hz, 6H).

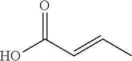

Choline 3-Butenoate 1:1 (CA3BE 1:1) ¹H NMR (400 MHz, d6-DMSO) δ 5.93 (ddt, J=17.3, 9.7, 7.3 Hz, 1H), 4.90-4.79 (m, 2H), 3.85 (h, J=2.6 Hz, 2H), 3.43 (dd, J=6.1, 3.8 Hz, 2H), 3.13 (s, 9H), 1.66 (dd, J=6.7, 1.8 Hz, 1H).

Choline 3-Butenoate 1:2 (CA3BE 1:2) ¹H NMR (400 MHz, d6-DMSO) δ 6.55 (dtd, J=13.7, 8.7, 7.7, 5.9 Hz, 1H), 6.02-5.69 (m, 2H), 5.02-4.89 (m, 2H), 3.84 (dq, J=5.4, 2.6 Hz, 2H), 3.46-3.37 (m, 2H), 3.12 (s, 9H), 1.75 (dd, J=6.9, 1.7 Hz, 4H).

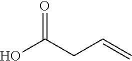

Choline Pentanoate 1:1 (CAPA 1:1) ¹H NMR (400 MHz, d6-DMSO) δ 3.85 (dq, J=8.0, 2.8 Hz, 2H), 3.46-3.34 (m, 2H), 3.13 (s, 9H), 1.91-1.81 (m, 2H), 1.39 (tt, J=7.8, 6.5 Hz, 2H), 1.29-1.15 (m, 2H), 0.83 (t, J=7.3 Hz, 3H).

Choline Pentanoate 1:2 (CAPA 1:2) ¹H NMR (400 MHz, d6-DMSO) δ 3.89-3.80 (m, 2H), 3.45-3.38 (m, 2H), 3.12 (s, 9H), 2.01 (t, J=7.4 Hz, 4H), 1.48-1.36 (m, 4H), 1.31-1.17 (m, 4H), 0.84 (t, J=7.3 Hz, 6H).

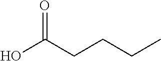

Choline 2-Pentenoate 1:1 (CA2PE 1:1) ¹H NMR (400 MHz, d6-DMSO) δ 6.34 (dtd, J=15.8, 6.4, 2.9 Hz, 1H), 5.63 (dd, J=15.5, 1.7 Hz, 1H), 3.84 (dq, J=5.9, 2.7 Hz, 2H), 3.46 (dd, J=6.2, 3.9 Hz, 2H), 3.15 (s, 9H), 2.08-1.96 (m, 2H), 0.93 (td, J=7.4, 2.0 Hz, 3H).

Choline 2-Pentenoate 1:2 (CA2PE 1:2) ¹H NMR (400 MHz, d6-DMSO) δ 6.61 (dt, J=15.3, 6.2 Hz, 2H), 5.75 (dt, J=15.5, 1.7 Hz, 2H), 3.85 (dq, J=5.4, 2.6 Hz, 2H), 3.44 (dd, J=6.1, 4.1 Hz, 2H), 3.14 (s, 9H), 2.10 (tt, J=9.1, 6.6 Hz, 4H), 0.97 (t, J=7.5 Hz, 6H).

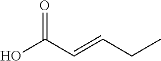

Choline 3-Pentenoate 1:1 (CA3PE 1:1) ¹H NMR (400 MHz, d6-DMSO) δ 5.58 (dt, J=14.1, 6.8 Hz, 1H), 5.32 (dq, J=13.3, 6.3 Hz, 1H), 3.89 (dq, J=5.8, 2.7 Hz, 2H), 3.51 (dd, J=6.5, 3.6 Hz, 2H), 3.21 (d, J=2.5 Hz, 9H), 2.72 (d, J=7.3 Hz, 2H), 1.63 (d, J=6.7 Hz, 3H).

Choline 3-Pentenoate 1:2 (CA3PE 1:2) ¹H NMR (400 MHz, d6-DMSO) δ 5.51 (dtd, J=13.9, 7.0, 1.7 Hz, 2H), 5.43-5.30 (m, 2H), 3.84 (td, J=5.6, 2.5 Hz, 2H), 3.44 (dt, J=7.1, 3.4 Hz, 2H), 3.13 (d, J=3.2 Hz, 9H), 2.80 (d, J=7.2 Hz, 4H), 1.60 (d, J=6.8 Hz, 6H).

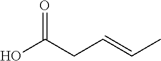

Choline Hexanoate 1:1 (CAHA 1:1) ¹H NMR (400 MHz, d6-DMSO) δ 3.84 (dt, J=7.4, 2.7 Hz, 2H), 3.49-3.42 (m, 2H), 3.16 (s, 9H), 1.87 (t, J=7.5 Hz, 2H), 1.41 (p, J=7.4 Hz, 2H), 1.27-1.15 (m, 4H), 0.83 (td, J=7.1, 1.8 Hz, 3H).

Choline Hexanoate 1:2 (CAHA 1:2) ¹H NMR (400 MHz, d6-DMSO) δ 3.84 (td, J=5.6, 2.6 Hz, 2H), 3.44 (dq, J=7.5, 3.7 Hz, 2H), 3.14 (dd, J=5.9, 2.8 Hz, 9H), 2.05 (dt, J=9.1, 4.5 Hz, 4H), 1.45 (p, J=7.3 Hz, 4H), 1.23 (pd, J=8.4, 7.9, 2.9 Hz, 8H), 0.88-0.80 (m, 6H).

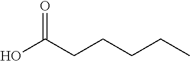

Choline 2-Hexenoate 1:1 (CA2HE 1:1) ¹H NMR (400 MHz, d6-DMSO) δ 6.27 (dt, J=15.4, 7.0 Hz, 1H), 5.62 (dd, J=15.4, 1.6 Hz, 1H), 3.89-3.81 (m, 2H), 3.50-3.43 (m, 2H), 3.15 (s, 9H), 1.99 (qd, J=7.1, 1.5 Hz, 2H), 1.35 (h, J=7.3 Hz, 2H), 0.86 (t, J=7.4 Hz, 3H).

Choline 2-Hexenoate 1:2 (CA2HE 1:2) ¹H NMR (400 MHz, d6-DMSO) δ 6.39-6.27 (m, 2H), 5.51 (d, J=15.5 Hz, 2H), 3.60 (dt, J=7.5, 3.5 Hz, 2H), 3.21 (dt, J=9.4, 5.2 Hz, 2H), 2.90 (d, J=6.4 Hz, 9H), 1.81 (q, J=7.3 Hz, 4H), 1.18-1.09 (m, 4H), 0.61 (td, J=7.4, 3.0 Hz, 6H).

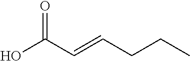

Choline 3-Hexenoate 1:1 (CA3HE 1:1) ¹H NMR (400 MHz, d6-DMSO) δ 5.56-5.48 (m, 1H), 5.29 (ddd, J=15.3, 7.2, 5.5 Hz, 1H), 3.84 (dq, J=5.6, 2.7 Hz, 2H), 3.49-3.42 (m, 2H), 3.15 (s, 9H), 2.67-2.61 (m, 2H), 1.94 (p, J=7.2 Hz, 2H), 0.91 (t, J=7.5 Hz, 3H).

Choline 3-Hexenoate 1:2 (CA3HE 1:2) ¹H NMR (400 MHz, d6-DMSO) δ 5.56-5.48 (m, 1H), 5.29 (ddd, J=15.3, 7.2, 5.5 Hz, 1H), 3.84 (dq, J=5.6, 2.7 Hz, 2H), 3.49-3.42 (m, 2H), 3.15 (s, 9H), 2.67-2.61 (m, 2H), 1.94 (p, J=7.2 Hz, 2H), 0.91 (t, J=7.5 Hz, 3H).

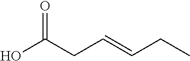

Choline trans-2-Methyl-2-Pentenoate 1:1 (CAGAS 1:1) ¹H NMR (400 MHz, d6-DMSO) δ 6.22 (td, J=7.3, 1.6 Hz, 1H), 3.85 (dq, J=7.7, 2.8 Hz, 2H), 3.49-3.42 (m, 2H), 3.15 (s, 9H), 1.98 (p, J=7.5 Hz, 2H), 1.64 (d, J=1.7 Hz, 3H), 0.91 (t, J=7.6 Hz, 3H).

Choline trans-2-Methyl-2-Pentenoate 1:2 (CAGAS 1:2) ¹H NMR (400 MHz, d6-DMSO) δ 6.43 (td, J=7.4, 1.8 Hz, 2H), 3.86 (td, J=5.6, 2.6 Hz, 2H), 3.49-3.40 (m, 2H), 3.15 (s, 9H), 2.06 (p, J=7.5 Hz, 4H), 1.69 (d, J=1.8 Hz, 6H), 0.95 (t, J=7.6 Hz, 6H).

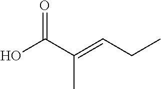

Choline Heptanoate 1:1 (CAHPA 1:1) ¹H NMR (400 MHz, d6-DMSO) δ 3.85 (dt, J=7.2, 2.7 Hz, 2H), 3.44-3.40 (m, 2H), 3.12 (s, 9H), 1.83 (td, J=7.5, 2.0 Hz, 2H), 1.39 (p, J=7.3 Hz, 2H), 1.20 (dt, J=7.9, 3.1 Hz, 5H), 0.85 (t, J=6.9 Hz, 3H).

Choline Heptanoate 1:2 (CAHPA 1:2) ¹H NMR (400 MHz, d6-DMSO) δ 3.84 (dq, J=5.4, 2.7 Hz, 2H), 3.43-3.38 (m, 2H), 3.11 (s, 9H), 2.02 (ddd, J=7.4, 5.9, 1.5 Hz, 4H), 1.42 (d, J=7.3 Hz, 5H), 1.31-1.17 (m, 13H), 0.85 (t, J=6.8 Hz, 6H).

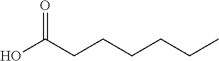

Choline 2-Heptenoate 1:1 (CA2HPE 1:1) ¹H NMR (400 MHz, d6-DMSO) δ 6.24 (dt, J=15.4, 6.9 Hz, 1H), 5.60 (dt, J=15.2, 1.5 Hz, 1H), 3.86 (td, J=5.6, 2.7 Hz, 2H), 3.49-3.42 (m, 2H), 3.15 (s, 9H), 2.06-1.96 (m, 2H), 1.30 (dtt, J=17.9, 7.4, 4.0 Hz, 4H), 0.86 (t, J=6.9 Hz, 3H).

Choline 2-Heptenoate 1:2 (CA2HPE 1:2) ¹H NMR (400 MHz, d6-DMSO) δ 6.60 (dt, J=15.7, 7.0 Hz, 2H), 5.81 (dt, J=15.4, 1.5 Hz, 2H), 3.92 (dq, J=5.5, 2.7 Hz, 2H), 3.54-3.47 (m, 2H), 3.20 (s, 9H), 2.15 (qd, J=7.0, 1.6 Hz, 4H), 1.49-1.29 (m, 8H), 0.94 (t, J=7.1 Hz, 6H).

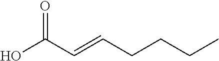

Choline 3-Heptenoate 1:1 (CA3HPE 1:1) ¹H NMR (400 MHz, d6-DMSO) δ 5.62-5.48 (m, 1H), 5.27-5.14 (m, 1H), 3.85 (dq, J=5.5, 2.7 Hz, 2H), 3.47-3.40 (m, 3H), 3.13 (s, 9H), 2.58 (dd, J=7.1, 1.5 Hz, 2H), 1.91 (q, J=7.5 Hz, 2H), 1.32 (h, J=7.4 Hz, 2H), 0.86 (t, J=7.3 Hz, 3H).

Choline 3-Heptenoate 1:2 (CA3HPE 1:2) ¹H NMR (400 MHz, d6-DMSO) δ 5.57-5.45 (m, 2H), 5.39-5.27 (m, 2H), 3.85 (td, J=5.6, 2.8 Hz, 2H), 3.45-3.38 (m, 2H), 3.12 (s, 9H), 2.75 (dd, J=7.0, 1.4 Hz, 3H), 1.93 (q, J=7.1 Hz, 3H), 1.33 (h, J=7.3 Hz, 4H), 0.86 (t, J=7.3 Hz, 6H).

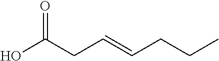

Choline Octanoate 1:1 (CAOA 1:1) ¹H NMR (400 MHz, d6-DMSO) δ 3.85 (dq, J=8.1, 2.8 Hz, 2H), 3.46-3.39 (m, 6H), 3.13 (s, 9H), 1.82 (t, J=7.4 Hz, 2H), 1.40 (q, J=7.3 Hz, 2H), 1.23 (qd, J=12.4, 11.2, 7.1 Hz, 9H), 0.86 (t, J=6.9 Hz, 3H).

Choline Octanoate 1:2 (CAOA 1:2) ¹H NMR (400 MHz, d6-DMSO) δ 3.65-3.56 (m, 2H), 3.25-3.18 (m, 2H), 2.90 (s, 9H), 1.80 (t, J=7.5 Hz, 4H), 1.20 (t, J=7.3 Hz, 4H), 1.03-0.95 (m, 16H), 0.59 (t, J=6.7 Hz, 6H).

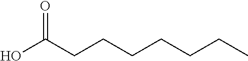

Choline 2-Octenoate 1:1 (CA2OE 1:1) ¹H NMR (400 MHz, d6-DMSO) δ 6.28 (dt, J=15.4, 6.9 Hz, 1H), 5.67-5.58 (m, 1H), 3.85 (dq, J=5.4, 2.6 Hz, 2H), 3.50-3.43 (m, 2H), 3.15 (s, 9H), 2.00 (qd, J=7.1, 1.5 Hz, 2H), 1.34 (p, J=7.1 Hz, 3H), 1.25 (tt, J=8.1, 5.1 Hz, 4H), 0.85 (t, J=6.9 Hz, 3H).

Choline 2-Octenoate 1:2 (CA2OE 1:2) ¹H NMR (300 MHz, d6-DMSO) δ 6.62-6.46 (m, 2H), 5.74 (dt, J=15.5, 1.6 Hz, 2H), 3.91-3.79 (m, 2H), 3.43 (dd, J=6.1, 4.0 Hz, 2H), 3.13 (s, 9H), 2.08 (qd, J=7.1, 1.5 Hz, 4H), 1.41-1.23 (m, 12H), 0.86 (t, J=6.7 Hz, 6H).

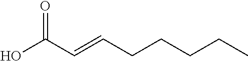

Choline 3-Octenoate 1:1 (CA3OE 1:1) ¹H NMR (400 MHz, d6-DMSO) δ 5.59-5.47 (m, 1H), 5.17 (dt, J=14.7, 6.7 Hz, 1H), 3.83 (dd, J=6.2, 3.7 Hz, 2H), 3.11 (s, 9H), 2.09 (s, 2H), 1.92 (d, J=6.9 Hz, 2H), 1.28 (q, J=5.1, 3.4 Hz, 4H), 0.90-0.82 (m, 3H).

Choline 3-Octenoate 1:2 (CA3OE 1:2) ¹H NMR (400 MHz, d6-DMSO) δ 5.50 (dt, J=14.5, 6.9 Hz, 2H), 5.35 (dt, J=15.3, 6.7 Hz, 2H), 3.84 (dq, J=5.7, 2.8 Hz, 2H), 3.44 (dd, J=7.6, 4.1 Hz, 2H), 3.17-3.11 (m, 9H), 2.80 (t, J=5.6 Hz, 4H), 1.95 (q, J=6.8 Hz, 4H), 1.27 (dq, J=11.3, 7.7, 5.5 Hz, 8H), 0.85 (td, J=7.2, 3.0 Hz, 6H).

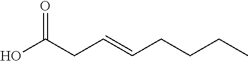

Choline Nonanoate 1:1 (CANA 1:1) ¹H NMR (400 MHz, d6-DMSO) δ 3.84 (dq, J=5.4, 2.6 Hz, 2H), 3.48-3.41 (m, 2H), 3.14 (s, 9H), 1.88 (t, J=7.5 Hz, 2H), 1.40 (t, J=7.2 Hz, 2H), 1.22 (q, J=6.1, 5.2 Hz, 10H), 0.85 (t, J=6.7 Hz, 3H).

Choline Nonanoate 1:2 (CANA 1:2) ¹H NMR (400 MHz, d6-DMSO) δ 3.89-3.80 (m, 2H), 3.45-3.37 (m, 2H), 3.12 (s, 9H), 2.02 (t, J=7.4 Hz, 4H), 1.43 (p, J=7.0 Hz, 5H), 1.23 (d, J=3.8 Hz, 22H), 0.90-0.81 (m, 6H).

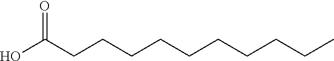

Choline 2-Nonenoate 1:1 (CA2NE 1:1) ¹H NMR (400 MHz, d6-DMSO) δ 6.23 (dt, J=15.7, 6.9 Hz, 1H), 5.60 (dd, J=15.3, 1.5 Hz, 1H), 3.86 (dq, J=7.8, 2.8 Hz, 2H), 3.50-3.43 (m, 2H), 3.16 (s, 9H), 2.00 (qd, J=7.1, 1.5 Hz, 2H), 1.33 (t, J=6.8 Hz, 2H), 1.26 (q, J=4.7 Hz, 6H), 0.90-0.82 (m, 3H).

Choline 2-Nonenoate 1:2 (CA2NE 1:2) ¹H NMR (400 MHz, d6-DMSO) δ 6.53 (dtd, J=17.6, 7.4, 3.0 Hz, 2H), 5.74 (dd, J=15.5, 1.7 Hz, 2H), 3.90-3.81 (m, 2H), 3.47-3.38 (m, 2H), 3.13 (d, J=2.1 Hz, 9H), 2.08 (qd, J=7.1, 1.5 Hz, 4H), 1.36 (q, J=7.1 Hz, 4H), 1.32-1.23 (m, 12H), 0.90-0.82 (m, 6H).

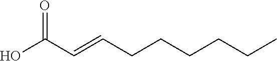

Choline 3-Nonenoate 1:1 (CA3NE 1:1) ¹H NMR (400 MHz, d6-DMSO) δ 5.54 (dtd, J=15.6, 7.1, 1.4 Hz, 1H), 5.28-5.16 (m, 1H), 3.84 (dq, J=5.5, 2.7 Hz, 2H), 3.47-3.40 (m, 2H), 3.14 (s, 9H), 2.59 (dd, J=7.1, 1.5 Hz, 2H), 1.92 (q, J=6.9 Hz, 2H), 1.36-1.18 (m, 6H), 0.86 (t, J=6.7 Hz, 3H).

Choline 3-Nonenoate 1:2 (CA3NE 1:2) ¹H NMR (400 MHz, d6-DMSO) δ 5.50 (dtd, J=15.1, 6.8, 1.3 Hz, 2H), 5.36 (ddd, J=15.3, 7.3, 5.9 Hz, 2H), 3.84 (dq, J=5.5, 2.6 Hz, 2H), 3.46-3.39 (m, 2H), 3.12 (s, 9H), 2.79 (dd, J=6.7, 1.5 Hz, 4H), 1.95 (q, J=6.9 Hz, 4H), 1.38-1.26 (m, 8H), 1.24 (d, J=6.4 Hz, 4H), 0.86 (t, J=6.8 Hz, 6H).

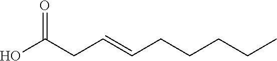

Choline Decanoate 1:1 (CADA 1:1) ¹H NMR (400 MHz, d6-DMSO) δ 3.87-3.81 (m, 2H), 3.46 (t, J=4.9 Hz, 2H), 3.16 (s, 9H), 1.90 (t, J=7.6 Hz, 2H), 1.40 (t, J=7.3 Hz, 2H), 1.20 (s, 12H), 0.82 (t, J=6.6 Hz, 3H).

Choline Decanoate 1:2 (CADA 1:2) ¹H NMR (400 MHz, d6-DMSO) δ 3.91 (dq, J=5.4, 2.6 Hz, 2H), 3.54-3.47 (m, 2H), 3.20 (s, 9H), 2.11 (t, J=7.5 Hz, 4H), 1.51 (p, J=7.0 Hz, 4H), 1.34 (s, 3H), 1.30 (s, 20H), 0.92 (t, J=6.8 Hz, 6H).

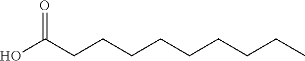

Choline 2-Decenoate 1:1 (CA2DE 1:1) ¹H NMR (400 MHz, d6-DMSO) δ 6.37 (dt, J=15.5, 6.9 Hz, 1H), 5.71 (d, J=15.3 Hz, 1H), 3.94 (dq, J=5.8, 2.6 Hz, 2H), 3.58-3.51 (m, 2H), 3.24 (s, 9H), 2.09 (q, J=7.1 Hz, 2H), 1.48-1.34 (m, 6H), 1.34 (s, 4H), 0.94 (t, J=6.8 Hz, 3H).

Choline 2-Decenoate 1:2 (CA2DE 1:2) ¹H NMR (400 MHz, d6-DMSO) δ 6.52 (dt, J=15.8, 6.9 Hz, 2H), 5.77-5.69 (m, 2H), 3.85 (p, J=2.8 Hz, 2H), 3.47-3.40 (m, 2H), 3.13 (s, 9H), 2.07 (q, J=7.1 Hz, 4H), 1.37 (p, J=6.8 Hz, 4H), 1.29 (d, J=8.3 Hz, 4H), 1.25 (s, 12H), 0.86 (t, J=6.6 Hz, 6H).

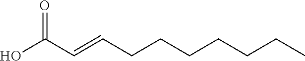

Choline 3-Decenoate 1:1 (CA3DE 1:1) ¹H NMR (400 MHz, d6-DMSO) δ 5.57-5.45 (m, 1H), 5.31-5.19 (m, 1H), 3.88-3.80 (m, 3H), 3.44-3.37 (m, 3H), 3.11 (s, 9H), 2.64 (dd, J=7.0, 1.4 Hz, 2H), 1.93 (q, J=6.6 Hz, 2H), 1.35-1.18 (m, 11H), 1.01-0.92 (m, 1H), 0.86 (h, J=3.1 Hz, 3H).

Choline 3-Decenoate 1:2 (CA3DE 1:2) ¹H NMR (400 MHz, d6-DMSO) δ 5.49 (dtt, J=15.1, 6.8, 1.3 Hz, 2H), 5.34 (dtt, J=14.9, 6.7, 1.4 Hz, 2H), 3.88-3.80 (m, 2H), 3.44-3.37 (m, 2H), 3.11 (s, 9H), 2.77 (dd, J=6.8, 1.3 Hz, 4H), 1.95 (q, J=6.4 Hz, 4H), 1.36-1.22 (m, 17H), 0.90-0.82 (m, 6H).

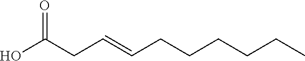

Choline Undecanoate 1:1 (CAUA 1:1) ¹H NMR (400 MHz, d6-DMSO) δ 3.95 (dq, J=5.8, 2.7 Hz, 2H), 3.60-3.53 (m, 2H), 3.26 (s, 9H), 1.99 (t, J=7.5 Hz, 2H), 1.55-1.47 (m, 2H), 1.32 (d, J=4.5 Hz, 14H), 0.94 (t, J=6.7 Hz, 3H).

Choline Undecanoate 1:2 (CAUA 1:2) ¹H NMR (400 MHz, d6-DMSO) δ 3.64-3.57 (m, 2H), 3.20 (d, J=4.8 Hz, 2H), 2.90 (s, 9H), 1.79 (t, J=7.5 Hz, 4H), 1.21 (q, J=7.0 Hz, 4H), 1.01 (s, 4H), 0.97 (s, 24H), 0.59 (t, J=6.7 Hz, 6H).

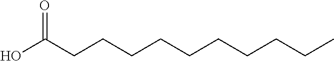

Choline 2-Undecenoate 1:1 (CA2UE 1:1) ¹H NMR (400 MHz, d6-DMSO) δ 6.36 (dt, J=14.5, 6.9 Hz, 1H), 5.71 (d, J=15.3 Hz, 1H), 4.00-3.91 (m, 2H), 3.65-3.55 (m, 2H), 3.28 (s, 9H), 2.09 (q, J=7.1 Hz, 2H), 1.47-1.35 (m, 6H), 1.34 (s, 6H), 0.94 (t, J=6.7 Hz, 3H).

Choline 2-Undecenoate 1:2 (CA2UE 1:2) ¹H NMR (400 MHz, d6-DMSO) δ 6.50 (dt, J=15.6, 6.9 Hz, 2H), 5.73 (dd, J=15.5, 1.7 Hz, 2H), 3.86 (dq, J=5.6, 2.8 Hz, 2H), 3.46 (dd, J=5.9, 4.1 Hz, 2H), 3.15 (s, 9H), 2.06 (q, J=7.2 Hz, 4H), 1.36 (p, J=6.9 Hz, 4H), 1.25 (q, J=6.5, 4.1 Hz, 20H), 0.85 (t, J=6.8 Hz, 6H).

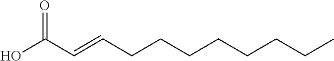

Choline Dodecanoate 1:1 (CADDA 1:1) ¹H NMR (400 MHz, d6-DMSO) δ 3.85 (q, J=2.9 Hz, 2H), 3.44 (dd, J=6.1, 3.8 Hz, 2H), 3.14 (s, 9H), 1.85 (t, J=7.5 Hz, 2H), 1.40 (p, J=7.1 Hz, 2H), 1.22 (d, J=8.4 Hz, 16H), 0.85 (t, J=6.6 Hz, 3H).

Choline Dodecanoate 1:2 (CADDA 1:2) ¹H NMR (400 MHz, d6-DMSO) δ 3.81 (dq, J=5.6, 2.7 Hz, 2H), 3.40 (dd, J=5.8, 4.2 Hz, 2H), 3.10 (s, 9H), 2.00 (t, J=7.5 Hz, 4H), 1.41 (q, J=7.1 Hz, 4H), 1.18 (s, 32H), 0.80 (t, J=6.7 Hz, 6H).

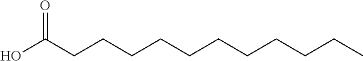

Choline Fumarate 1:1 (CAFU 1:1) ¹H NMR (400 MHz, d6-DMSO) δ 6.46 (s, 2H), 3.84 (dq, J=5.3, 2.6 Hz, 2H), 3.48-3.37 (m, 3H), 3.12 (s, 9H).

Choline Fumarate 1:2 (CAFU 1:2) ¹H NMR (400 MHz, d6-DMSO) δ 6.55 (s, 4H), 3.84 (dq, J=5.3, 2.6 Hz, 2H), 3.41 (dd, J=6.0, 4.1 Hz, 2H), 3.11 (s, 9H).

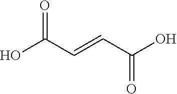

Choline Malonate 1:1 (CAMA 1:1) ¹H NMR (400 MHz, d6-DMSO) δ 3.84 (td, J=5.7, 2.7 Hz, 2H), 3.41 (t, J=5.2 Hz, 2H), 3.12 (s, 9H), 2.77 (s, 2H).

Choline Malonate 1:2 (CAMA 1:2) ¹H NMR (400 MHz, d6-DMSO) δ 3.84 (td, J=5.8, 2.7 Hz, 2H), 3.44-3.36 (m, 2H), 3.13 (s, 9H), 2.93 (s, 4H).

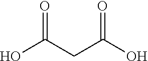

Choline Maleate 1:1 (CAME 1:1) ¹H NMR (400 MHz, d6-DMSO) δ 6.12 (d, J=2.6 Hz, 2H), 3.86 (td, J=5.7, 2.7 Hz, 2H), 3.43 (t, J=5.3 Hz, 2H), 3.13 (s, 9H).

Choline Maleate 1:2 (CAME 1:2) ¹H NMR (400 MHz, d6-DMSO) δ 6.16 (s, 4H), 3.84 (dq, J=5.3, 2.6 Hz, 2H), 3.47-3.35 (m, 2H), 3.13 (s, 9H).

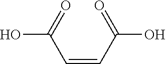

Choline Malate 1:1 (CAMI 1:1) ¹H NMR (400 MHz, d6-DMSO) δ 7.07 (s, 1H), 3.93 (dd, J=9.1, 4.6 Hz, 1H), 3.84 (td, J=5.7, 2.7 Hz, 2H), 3.45-3.38 (m, 2H), 3.11 (s, 9H), 2.55 (dd, J=15.6, 9.1 Hz, 1H), 2.31 (dd, J=15.5, 4.6 Hz, 1H).

Choline Malate 1:2 (CAMI 1:2) ¹H NMR (400 MHz, d6-DMSO) δ 7.87 (s, 2H), 4.16 (dd, J=7.1, 5.6 Hz, 2H), 3.82 (dq, J=7.7, 2.6 Hz, 2H), 3.45-3.34 (m, 2H), 3.08 (d, J=8.1 Hz, 9H), 2.61 (dd, J=15.6, 5.5 Hz, 2H), 2.39 (dd, J=15.6, 7.2 Hz, 2H).

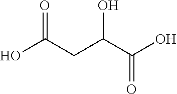

Choline Acetoxyacetate 1:1 (CAAA 1:1) ¹H NMR (400 MHz, d6-DMSO) δ 4.16 (s, 2H), 3.83 (h, J=2.6 Hz, 2H), 3.47-3.37 (m, 2H), 3.13 (s, 9H), 1.99 (s, 3H).

Choline Acetoxyacetate 1:2 (CAAA 1:2) ¹H NMR (400 MHz, d6-DMSO) δ 4.30 (s, 4H), 3.76 (s, 2H), 3.42 (s, 2H), 3.11 (s, 9H), 2.03 (s, 6H).

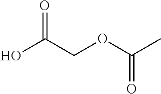

Choline Ethoxyacetate 1:1 (CAEA 1:1) ¹H NMR (400 MHz, d6-DMSO) δ 3.89-3.82 (m, 2H), 3.44 (s, 2H), 3.41 (s, 2H), 3.39 (s, 2H), 3.13 (s, 9H), 1.06 (t, J=7.0 Hz, 3H).

Choline Ethoxyacetate 1:2 (CAEA 1:2) ¹H NMR (400 MHz, d6-DMSO) δ 3.85 (dq, J=8.0, 2.8 Hz, 2H), 3.77 (s, 4H), 3.44 (d, J=7.2 Hz, 4H), 3.41 (d, J=7.0 Hz, 2H), 3.14 (s, 9H), 1.07 (t, J=7.1 Hz, 6H).

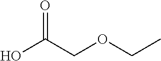

Choline 3-Mercaptopropionate 1:1 (CA3MP 1:1) ¹H NMR (400 MHz, d6-DMSO) δ 3.82 (dq, J=7.8, 2.8 Hz, 2H), 3.43 (dt, J=5.0, 3.3 Hz, 2H), 3.13 (d, J=4.0 Hz, 9H), 2.53 (t, J=7.1 Hz, 2H), 2.21 (t, J=7.1 Hz, 2H).

Choline 3-Mercaptopropionate 1:2 (CA3MP 1:2) ¹H NMR (400 MHz, d6-DMSO) δ 3.84 (s, 2H), 3.45-3.34 (m, 2H), 3.12 (d, J=4.7 Hz, 9H), 2.58 (dd, J=8.6, 4.9 Hz, 4H), 2.39 (q, J=6.9, 6.3 Hz, 4H).

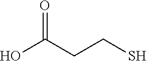

### Example 3: Results and Discussion

Synthesis and Characterization of ILs. In order to assess the antibacterial efficacy of ILs, a large library of choline carboxylic-acid based ILs was synthesized. To accomplish this, cation and anion were combined in either 1:1 or 1:2 mole ratios via a metathesis reaction in which the choline bicarbonate was added dropwise to the anion on a stir plate, liberating carbon dioxide and water upon deprotonation of the carboxylic acid anion. After stirring for 24 hours, the IL was moved to a rotary evaporator and then a vacuum oven to remove any excess water. NMR spectroscopy showed proton peaks and integration consistent with the expected identity and mole ratios of the ILs, which suggested successful synthesis. NMR characterizations and anion structures are listed in Example 2.

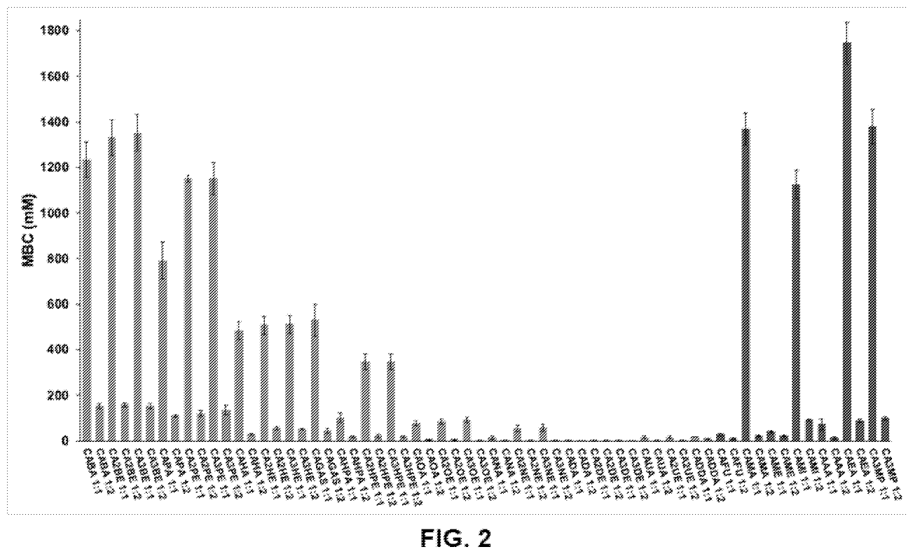

Antimicrobial Effects Enhanced by Combination of Cation and Anion. While maintaining a constant cation, ILs with a range of different anions were used to study the effects of mole ratio, saturation and placement of a double bond, chain length, and the presence of chalcogenic substituents on bactericidal activity. IL solutions were prepared as described above and added to liquid E. coli culture at varying concentrations to determine the minimum concentration of each cation-anion combination that completely eradicated growth on LBKan agar plates. ILs with the lowest MBC values were interpreted as the best candidates for E. coli bactericide (FIG. 2). From left to right on the x-axis, chain length of the anion is increased from 4-12 carbons with each anion being tested in first a 1:1 then 1:2 molar ratio. The anions with chalcogenic species appear on the far right of the figure (dark grey bars). In all scenarios, doubling the presence of the anion significantly decreases the MBC by at least one order of magnitude. Additional E. coil MBC values are presented in Table 1:

TABLE 1
E. coli MBC Values

| | | | E. coli | |
|---|---|---|---|---|
| | | Abbrevi- | MBC | |
| | IL Name | ation | (mM) | ± |
| 1 | Choline Butanoate 1:1 | CABA 1:1 | 1234.35 | 76.34 |
| 2 | Choline Butanoate 1:2 | CABA 1:2 | 152.70 | 10.57 |
| 3 | Choline 2-Butenoate 1:1 | CA2BE 1:1 | 1330.40 | 79.50 |
| 4 | Choline 2-Butenoate 1:2 | CA2BE 1:2 | 158.97 | 9.35 |
| 5 | Choline 3-Butenoate 1:1 | CA3BE 1:1 | 1350.93 | 80.66 |
| 6 | Choline 3-Butenoate 1:2 | CA3BE 1:2 | 153.33 | 10.62 |
| 7 | Choline Pentanoate 1:1 | CAPA 1:1 | 792.10 | 81.27 |
| 8 | Choline Pentanoate 1:2 | CAPA 1:2 | 110.37 | 4.79 |
| 9 | Choline 2-Pentenoate 1:1 | CA2PE 1:1 | 1151.30 | 14.90 |
| 10 | Choline 2-Pentenoate 1:2 | CA2PE 1:2 | 120.10 | 12.83 |
| 11 | Choline 3-Pentenoate 1:1 | CA3PE 1:1 | 1152.23 | 71.25 |
| 12 | Choline 3-Pentenoate 1:2 | CA3PE 1:2 | 134.83 | 20.74 |
| 13 | Choline Hexanoate 1:1 | CAHA 1:1 | 483.53 | 39.89 |
| 14 | Choline Hexanoate 1:2 | CAHA 1:2 | 28.23 | 4.45 |
| 15 | Choline 2-Hexenoate 1:1 | CA2HE 1:1 | 507.63 | 39.95 |
| 16 | Choline 2-Hexenoate 1:2 | CA2HE 1:2 | 54.37 | 7.75 |
| 17 | Choline 3-Hexenoate 1:1 | CA3HE 1:1 | 511.17 | 40.24 |
| 18 | Choline 3-Hexenoate 1:2 | CA3HE 1:2 | 52.00 | 4.50 |
| 19 | Choline trans-2-Methyl-2- | CAGAS 1:1 | 530.73 | 69.25 |
| | Pentenoate 1:1 | | | |
| 20 | Choline trans-2-Methyl-2- | CAGAS 1:2 | 44.60 | 12.01 |
| | Pentenoate 1:2 | | | |
| 21 | Choline Heptanoate 1:1 | CAHPA 1:1 | 101.80 | 19.57 |
| 22 | Choline Heptanoate 1:2 | CAHPA 1:2 | 16.33 | 4.04 |
| 23 | Choline 2-Heptenoate 1:1 | CA2HPE 1:1 | 346.93 | 34.52 |
| 24 | Choline 2-Heptenoate 1:2 | CA2HPE 1:2 | 21.33 | 6.80 |
| 25 | Choline 3-Heptenoate 1:1 | CA3HPE 1:1 | 347.63 | 34.62 |
| 26 | Choline 3-Heptenoate 1:2 | CA3HPE 1:2 | 18.93 | 4.10 |
| 27 | Choline Octanoate 1:1 | CAOA 1:1 | 76.63 | 10.95 |
| 28 | Choline Octanoate 1:2 | CAOA 1:2 | 4.91 | 2.12 |
| 29 | Choline 2-Octenoate 1:1 | CA2OE 1:1 | 84.70 | 10.60 |
| 30 | Choline 2-Octenoate 1:2 | CA2OE 1:2 | 5.18 | 2.25 |
| 31 | Choline 3-Octenoate 1:1 | CA3OE 1:1 | 92.00 | 12.30 |
| 32 | Choline 3-Octenoate 1:2 | CA3OE 1:2 | 2.16 | 0.38 |
| 33 | Choline Nonanoate 1:1 | CANA 1:1 | 13.63 | 5.95 |
| 34 | Choline Nonanoate 1:2 | CANA 1:2 | 1.01 | 0.35 |
| 35 | Choline 2-Nonenoate 1:1 | CA2NE 1:1 | 56.40 | 15.22 |
| 36 | Choline 2-Nonenoate 1:2 | CA2NE 1:2 | 1.22 | 0.61 |
| 37 | Choline 3-Nonenoate 1:1 | CA3NE 1:1 | 57.50 | 15.48 |
| 38 | Choline 3-Nonenoate 1:2 | CA3NE 1:2 | 1.02 | 0.35 |
| 39 | Choline Decanoate 1:1 | CADA 1:1 | 3.48 | 0.54 |
| 40 | Choline Decanoate 1:2 | CADA 1:2 | 0.56 | 0.00 |
| 41 | Choline 2-Decenoate 1:1 | CA2DE 1:1 | 3.67 | 0.58 |
| 42 | Choline 2-Decenoate 1:2 | CA2DE 1:2 | 0.77 | 0.34 |
| 43 | Choline 3-Decenoate 1:1 | CA3DE 1:1 | 3.10 | 1.07 |
| 44 | Choline 3-Decenoate 1:2 | CA3DE 1:2 | 0.57 | 0.00 |
| 45 | Choline Undecanoate 1:1 | CAUA 1:1 | 15.50 | 5.36 |
| 46 | Choline Undecanoate 1:2 | CAUA 1:2 | 1.98 | 0.29 |
| 47 | Choline 2-Undecenoate 1:1 | CA2UE 1:1 | 15.08 | 5.23 |
| 48 | Choline 2-Undecenoate 1:2 | CA2UE 1:2 | 1.78 | 0.61 |
| 49 | Choline Dodecanoate 1:1 | CADDA 1:1 | 17.10 | 0.00 |
| 50 | Choline Dodecanoate 1:2 | CADDA 1:2 | 8.34 | 2.88 |
| 51 | Choline Fumarate 1:1 | CAFU 1:1 | 27.17 | 6.70 |
| 52 | Choline Fumarate 1:2 | CAFU 1:2 | 10.05 | 4.37 |
| 53 | Choline Malonate 1:1 | CAMA 1:1 | 1368.53 | 69.69 |
| 54 | Choline Malonate 1:2 | CAMA 1:2 | 21.60 | 4.68 |
| 55 | Choline Maleate 1:1 | CAME 1:1 | 38.37 | 6.70 |
| 56 | Choline Maleate 1:2 | CAME 1:2 | 20.00 | 4.33 |
| 57 | Choline Malate 1:1 | CAMI 1:1 | 1126.13 | 60.91 |
| 58 | Choline Malate 1:2 | CAMI 1:2 | 91.80 | 3.98 |
| 59 | Choline Acetoxyacetate 1:1 | CAAA 1:1 | 73.40 | 24.09 |
| 60 | Choline Acetoxyacetate 1:2 | CAAA 1:2 | 12.41 | 4.31 |
| 61 | Choline Ethoxyacetate 1:1 | CAEA 1:1 | 1746.07 | 91.28 |
| 62 | Choline Ethoxyacetate 1:2 | CAEA 1:2 | 89.20 | 8.10 |
| 63 | Choline 3-Mercaptopropionate 1:1 | CA3MP 1:1 | 1378.42 | 74.62 |
| 64 | Choline 3-Mercaptopropionate 1:2 | CA3MP 1:2 | 101.07 | 8.45 |

Those ILs with oxygen or sulfur species were not particularly efficacious inhibitors of bacterial growth regardless of mole ratio in relation to the top candidates, so the focus was shifted to the non-substituted linear alkyl chains. Previous studies have shown that both choline and longer chain carboxylic acids have antibacterial properties individually. However, the effect is magnified when ions of the two compounds are combined to form an IL. When comparing the individual reagents of the IL, choline shows bactericidal activity around 889 mM and lauric acid around 1562.5 mM, while choline dodecanoate (CADDA) 1:1, which is comprised of one choline and one lauric acid moiety, has killing efficacy at concentrations as low as 17.1 mM.

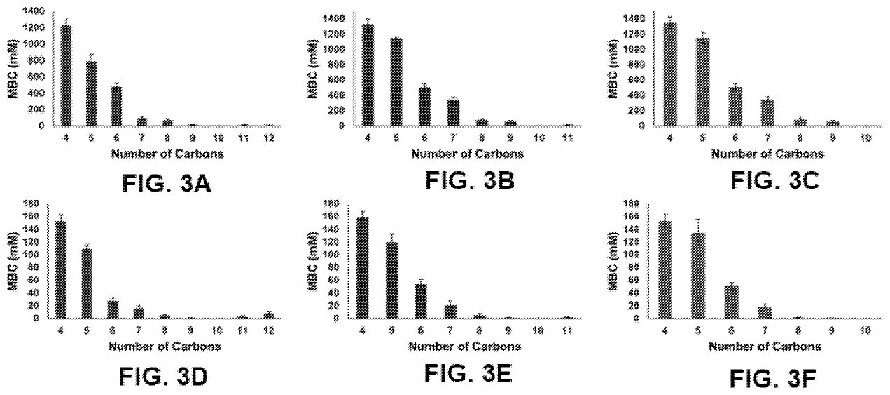

When comparing anions with chains ranging in lengths of 4-12 carbons, there is an increase in inhibitive activity in chain lengths of 8-12 because of increasing hydrophobic and chaotropic interactions with the bacterial membranes. Relating 1:1 and 1:2 mole ratios of choline to commercially available saturated and unsaturated 2-position and 3-position anions, there is roughly the same trend, however, increasing the mole ratio to 1:2 decreases the MBC values for even the 4 carbon chains by an order of magnitude because of the excess anion's availability to interact with the biological membranes (FIGS. 3A-3F). However, saturation and placement of the double bond affected the bactericidal efficacy within chains of the same length because the absence of π bonds leads to greater flexibility of the anion alkyl tail. Therefore, the anionic species is able to move more freely and more easily perturb the bacterial membranes at different locations.

For the 1:1 molar ratios, the general progression for MBC was saturated <2-position unsaturated <3-position unsaturated. However, for the 1:2 ILs, the relative trend for MBC was saturated ≤3-position unsaturated <2-position unsaturated. Since 1:1 and 1:2 ILs see the saturated anions as most efficacious, it is believed that that the reduced rigidity of those anions allows them to enter the membranes more freely.

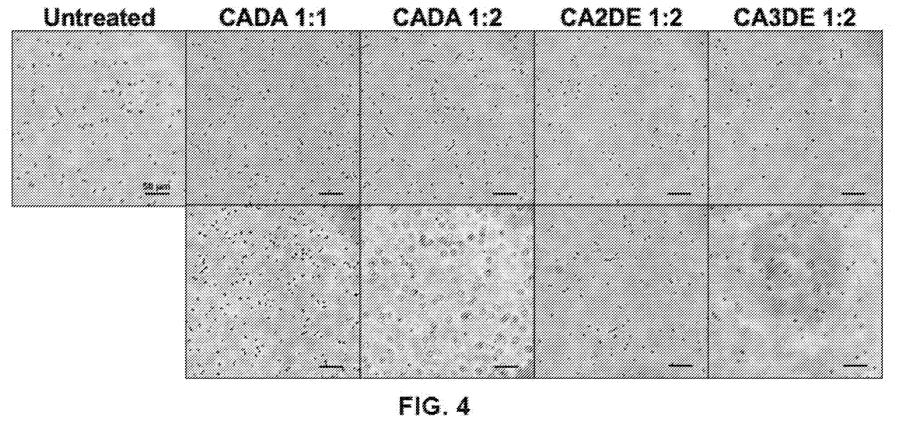

Brightfield Microscopy Images. To grasp a better understanding of the antimicrobial mechanism of the ILs, E. coli cells were dosed with lethal (MBC concentration) and sublethal (one-half MBC concentration) treatment, and were imaged by brightfield microscopy. Compared to the untreated cells, the microscopy shows that when the sublethal dose is applied, the IL begins interacting with the lipids in the biological membrane launching the formation of a coating around the cells. However, when the lethal treatment is given, there is a clear layer of IL encompassing the E. coli cells and debris is seen within the field of view, meaning that the IL is initiating bactericide by surrounding, “attacking,” and disrupting the membranes (FIG. 4). When millimolar quantities of the IL is added to the bacteria in solution, approximately 10²⁰ ions are produced, which then coat the micron-sized bacterium.

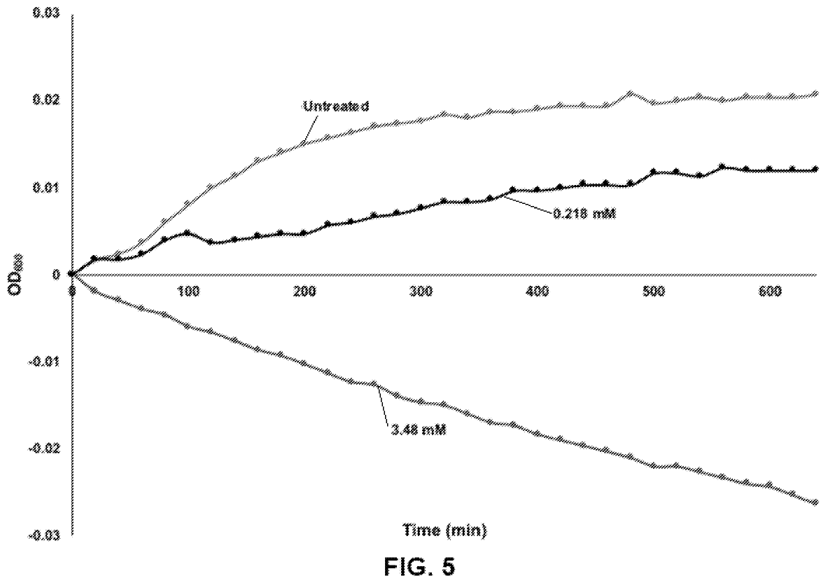

Bactericidal and Bacteriostatic Activity Kinetics. Time-dependent studies were conducted over a period of 16 hours with lethal and sublethal concentrations of IL to determine if bacteriostatic concentrations still inhibit the growth of E. coli even though complete eradication does not occur. A 1% (w/v) solution of CADA 1:1, which corresponds to the MBC of the IL when added to culture at 1% (v/v), was serially diluted by 2-fold down to one-sixteenth of the initial concentration. CADA 1:1 was employed instead of CADA 1:2 in this study because of its lowered turbidity in comparison. 180 μL of liquid E. coli culture was added to each well of a 96-well plate and brought up to 200 μL with either IL treatment or growth media alone if untreated. FIG. 5 shows OD₆₀₀ values versus time starting at the onset of the logarithmic phase of the untreated cell growth. The untreated cells grew logarithmically as expected, while cells treated with lethal concentrations were inhibited immediately with a steady decrease in OD₆₀₀. However, the cells treated with sublethal concentrations of IL still show what seem to be minor inhibitory interactions with the IL before exiting the lag phase and starting to grow. Since the lag phase was extended in the cells treated with sublethal doses, the E. coli did not begin normal growth until about two hours after the untreated cells entered the log phase. The turbidity of longer chain choline carboxylic acid-based ILs is quite high in aqueous solution and can falsely be determined as growth in OD measurements. Previous studies have shown that the critical micellar concentration (CMC) tends to increase with alkyl chain length and ionic strength, which could potentially affect antimicrobial efficacy. Therefore, IL solutions in sterile water at the same concentrations used to treat the bacteria were utilized as a control and subtracted from the wells with E. coli cells receiving IL treatment. Upon subtraction, data from sublethally treated wells slowly increased over time, while the opposite was seen immediately for the lethally treated cells. This shows that even miniscule concentrations of IL affected the normal growth of E. coli, with a linear proportionality in the concentration of IL and the level of inhibition that occurred. This suggests that even far below the MBC, ILs offer a way to inhibit the growth of bacteria.

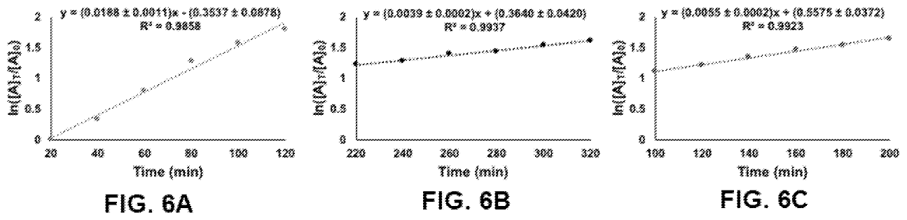

Integration of Growth Inhibition Following First-Order Kinetics. Once raw OD₆₀₀ measurements were recorded, first-order kinetic integration was performed to show that the inhibition of growth follows first-order kinetics. This was achieved by plotting In([A]_{T}/[A]₀) vs. time during the growth period to produce a linear representation of the logarithmic growth. Once the integrations were performed and plotted, rate constants were extracted with uncertainty from the slope of the trendlines. For the untreated cells and cells treated with sublethal concentrations, the logarithmic growth was positive, therefore, the rate constant should be positive. However, the cells treated with lethal concentrations exhibited immediate inhibition or negative growth, so the rate constant must be negative as well (FIGS. 6A-6D). When comparing the magnitude of the rate constants, the cells that received sublethal treatment (k=0.0039±0.0002) grew at about 20-25% of the rate of the untreated cells (k=0.0188±0.0011) suggesting that interactions between the IL moieties and E. coli were hindering the growth rate. The rate of inhibition (k=0.0055±0.0002) was almost one-third of the growth rate of the cells that received no treatment, meaning that the inhibition is occurring immediately and for the duration of the study, suppressing the natural growth of the cells by 70%.

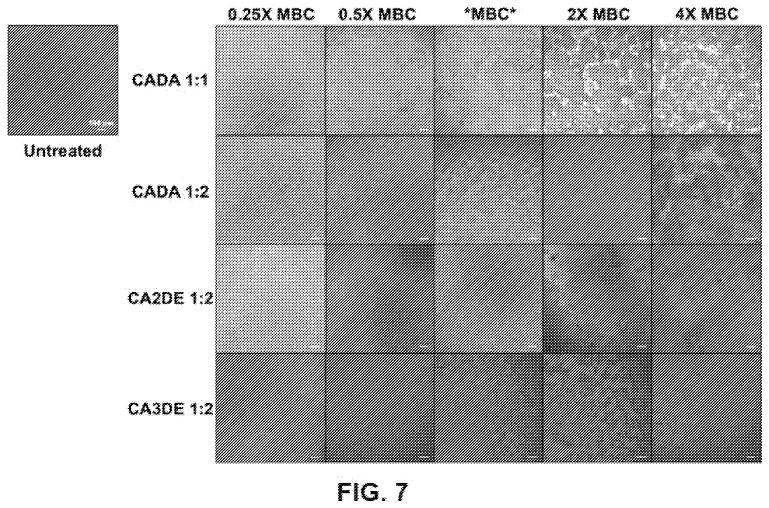

Human Biocompatibility Studies. Since choline carboxylic acid ILs are composed of biocompatible materials, they were tested to determine if their ionic form caused adverse effects to human cells at the concentrations needed to eradicate bacteria. HEK-293 cells were cultured to an initial concentration of 3×10⁴ cells/well. Once the cells reached 80% confluency, they were treated with CADA 1:1, CADA 1:2, CA2DE 1:2, and CA3DE 1:2 at concentrations of one-fourth, one-half, equal to, two times, and four times the MBC found for each IL and incubated for 24 hours. Before the addition of the viability testing reagent, brightfield microscopy images were obtained for untreated cells as well as each concentration for all four ILs to determine a qualitative maximum IL concentration that can be introduced to mammalian cells with minimal adverse effects. Previous studies suggested that extending the alkyl chain length on either the anion or cation increases cytotoxicity. However, the chain length of the anion and MBC were inversely related for E. coli until reaching the peak at n=10 in this study. Here, all four ILs contained similar constituents so the results were comparable (FIG. 7). With each IL, there was maintainable viability of the cells with concentrations at and below the MBC. Conversely, the viability of the cells was dramatically decreased once the MBC concentrations were doubled and even more so once they were quadrupled. At concentrations above the MBC, necrosis occurs in almost half of the cellular population while the cells that remain no longer appear healthy.

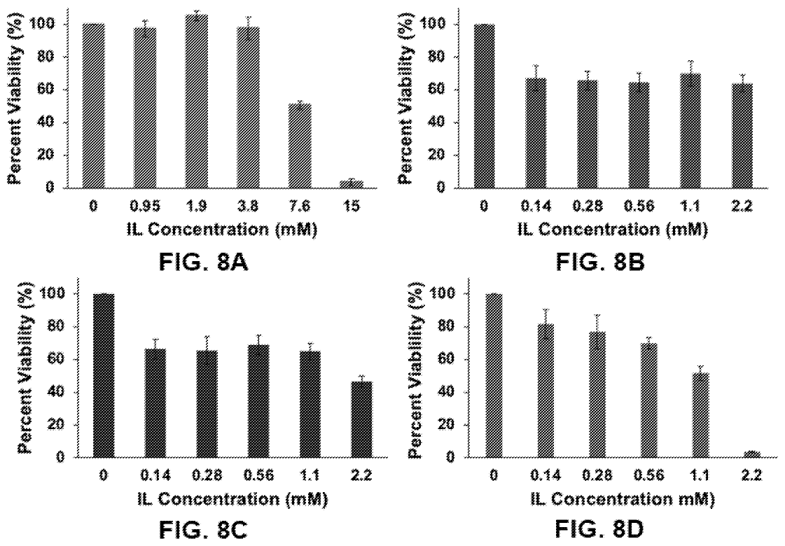

The viability of the cells was then quantified by preparing the IL-treated cells with 100 μL of CellTiter-Glo(R) Luminescent Cell Viability reagent in each well and measuring the luminescence on the plate reader. This viability assay determines the number of live cells present based on the ATP quantitation and did not require cell washing and removal of the cell medium. CellTiter-Glo(R) was chosen over the conventional MTT assay because the MTT assay can be prone to overestimating the number of viable cells compared to measurement of the ATP levels. The results are expressed as percent viability and present as mean±standard deviation of three independent experiments (FIGS. 8A-8D). With each IL, the quantitative results showed minimal toxic effects at concentrations at or below the MBC, which was comparable to the outcomes seen in the images above. When comparing percent viabilities throughout the first three treatments, CADA 1:1 excelled with over 97% viability, while the other three candidates conserved at least 65% even at the MBC. Significant decreases in viability were not seen until the MBC was multiplied by two or four, however, CADA 1:2 maintained good viability (>60%) across all five concentrations (FIG. 8B). This means that if treatment were applied to bacteria in a human host at the MBC, it is likely that minimal damage would be caused to the mammalian cells.

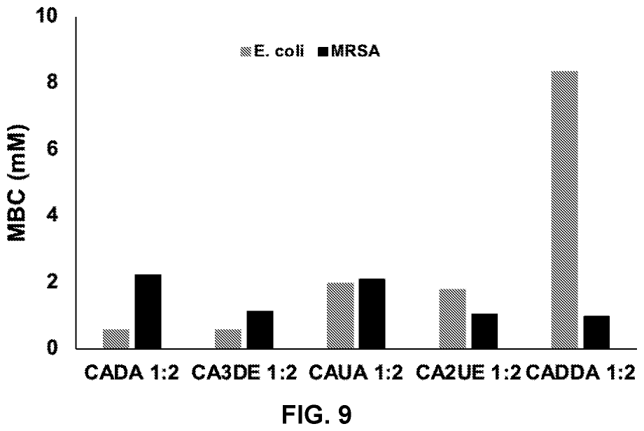

Comparative Inhibition of Gram-Negative and Gram-Positive Bacteria. Bacterial strains, such as MRSA, that possess antibiotic-resistant properties are challenging to treat. Previous studies have shown that bulky salts that can engage in electrostatic interactions with the biological membrane increase the antimicrobial activity of ILs against resistant strains. Here, 1:2 molar ratio ILs with saturated and unsaturated anions of chain lengths 8-12 were added to cultures of MRSA to generate MBC values. Data was inconclusive for n=8 and n=9, however, there were significant similarities in lethal concentrations for n=10 and n=11. Even so, the Gram-positive bacteria saw a peak in MBC of 0.99 mM at n=12, whereas the Gram-negative species saw a peak of 0.56 mM at n=10 (FIG. 9). As mentioned previously, the peptidoglycan layer of Gram-negative species is much thinner than Gram-positive and could potentially be why the Gram-negative species are eradicated by ILs with shorter chained anions. According to previous literature, ILs have shown a duality in antimicrobial mechanisms by utilizing both the cation and the anion to enhance the antiseptic effect. In such cases, the cation binds to the negatively-charged polymers in the peptidoglycan while the anion participates in reprotonation in acidic surroundings or in hydrophobic interactions depending on the environment. When comparing saturation, MRSA is more greatly affected by unsaturated chains, which is the opposite of what was seen in E. coli. In both cases, the MBC was still much lower than literature values for either choline or the anion alone, meaning that the induced interactions caused by introducing the charged solvents into the biological system are greater than hydrophobic interactions alone. Additional results are presented in Table 2.

TABLE 2
Comparative MBC Values for E. coli and MRSA

| | | E. coli | MRSA MBC |
|---|---|---|---|
| IL Name | Abbreviation | MBC (mM) | (mM) |
| Choline Decanoate 1:2 | CADA 1:2 | 0.56 | 2.23 |
| Choline 3-Decanoate 1:2 | CA3DE 1:2 | 0.57 | 1.13 |
| Choline Undecanoate 1:2 | CAUA 1:2 | 1.98 | 2.10 |
| Choline 2-Undecanoate 1:2 | CA2UE 1:2 | 1.78 | 1.06 |
| Choline Dodecanoate 1:2 | CADDA 1:2 | 8.34 | 0.99 |

Cost-Effectiveness of Ionic Liquid Antimicrobial Treatments. Underuse of some antiseptics tends to occur because of economic impact, so the cost of the production of ILs as antimicrobial agents was calculated. Commercially available prices of starting materials (from Sigma-Aldrich) were utilized to calculate the cost of 1 mole of choline and 2 moles of decanoic acid. Once found, the price per mole was multiplied by the MBC of CADA 1:2, and the totals were added together. Therefore, it would cost about $0.11 to produce 1 liter of CADA 1:2 at the MBC for this strain of E. coli. Note that for a commercial scale, this number is likely to decrease further. When compared to the cost of buying common household or laboratory antiseptics, the production of ILs is 10-100-fold cheaper depending on the comparable antimicrobial agent.

Conclusion: The increasing threat of antibiotic resistance globally necessitates the urgent development of safe, effective, and affordable antimicrobial materials. The main goal of this investigation was to develop ILs that could similarly affect both Gram-positive and Gram-negative bacteria while inducing minimal damage to human cells. By manipulating the anion and mole ratio of the ILs, an inverse proportionality between anion chain length and MBC was discerned for both Gram-negative and Gram-positive bacteria. The best performing ILs, choline decanoate 1:2 (CADA 1:2) and choline dodecanoate 1:2 (CADDA 1:2), had an MBC of 0.56 mM for E. coli and 0.99 mM for MRSA, respectively. It was observed that the Gram-positive bacteria required longer chained anions to disrupt their thicker peptidoglycan layered cell envelopes. Through microscopic imaging, the interactions between ILs and the outer structures of E. coli membranes at sublethal and lethal concentrations of IL were visualized. Time-dependent growth studies illustrated a linear relationship between IL treatment concentration and growth inhibition determining that at sublethal concentrations much lower than the MBC the lag phase is extended while at the lethal concentration immediate inhibition or bactericide occurs. Overall, this study illustrates that long-chain choline carboxylic acid ILs are viable as human-safe, economical, and effective antimicrobial agents.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Agatemor, C.; et al. Ionic Liquids for Addressing Unmet Needs in Healthcare. Bioengineering & Translational Medicine 2018, 3 (1), 7-25.
2. Benedetto, A. Room-Temperature Ionic Liquids Meet Bio-Membranes: The State-of-the-Art. Biophys Rev 2017, 9 (4), 309-320.
3. Benedetto, A.; et al. Room-Temperature Ionic Liquids and Biomembranes: Setting the Stage for Applications in Pharmacology, Biomedicine, and Bionanotechnology. Langmuir 2018, 34 (33), 9579-9597.
4. Bhattacharya, G.; et al. Thermodynamics of Interaction of Ionic Liquids with Lipid Monolayer. Biophys Rev 2018, 10 (3), 709-719.
5. Bingham, R. J.; et al. Computational Study of Room-Temperature Ionic Liquids Interacting with a POPC Phospholipid Bilayer. J Phys Chem B 2012, 116 (36), 11205-11216.
6. Borkowski, A.; et al. Different Antibacterial Activity of Novel Theophylline-Based Ionic Liquids—Growth Kinetic and Cytotoxicity Studies. Ecotoxicol Environ Saf 2016, 130, 54-64.
7. Borkowski, A.; et al. Interaction of Quaternary Ammonium Ionic Liquids with Bacterial Membranes-Studies with Escherichia Coli R1-R4-Type Lipopolysaccharides. Journal of Molecular Liquids 2017, 246, 282-289.
8. Cho, C.-W.; et al. Modelling for Antimicrobial Activities of Ionic Liquids towards Escherichia Coli, Staphylococcus Aureus and Candida Albicans Using Linear Free Energy Relationship Descriptors. J Hazard Mater 2016, 311, 168-175.
9. Coleman, D.; et al. Antimicrobial Toxicity Studies of Ionic Liquids Leading to a ‘Hit’ MRSA Selective Antibacterial Imidazolium Salt. Green Chem 2012, 14 (5), 1350-1356.
10. Cook, K.; et al. Correlating Lipid Membrane Permeabilities of Imidazolium Ionic Liquids with Their Cytotoxicities on Yeast, Bacterial, and Mammalian Cells. Biomolecules 2019, 9 (6), 251.
11. Curreri, A. M.; et al. Recent Advances in Ionic Liquids in Biomedicine. Advanced Science 2021, 8 (17), 2004819.
12. de Leo, F.; et al. Testing the Antimicrobial Properties of an Upcoming “Environmental-Friendly” Family of Ionic Liquids. J Mol Liq 2017, 248, 81-85.
13. Docherty, K. M.; et al. Toxicity and Antimicrobial Activity of Imidazolium and Pyridinium Ionic Liquids. Green Chem 2005, 7 (4), 185-189.
14. Duman, A. N.; et al. Synthesis of New Water-Soluble Ionic Liquids and Their Antibacterial Profile against Gram-Positive and Gram-Negative Bacteria. Heliyon 2019, 5 (10), e02607-e02607.
15. Egorova, K. S.; et al. Biological Activity of Ionic Liquids and Their Application in Pharmaceutics and Medicine. Chemical Reviews 2017, 117 (10), 7132-7189.
16. Ferraz, R.; et al. Ionic Liquids as Active Pharmaceutical Ingredients. ChemMedChem 2011, 6 (6), 975-985.
17. Ferraz, R.; et al. Synthesis and Antibacterial Activity of Ionic Liquids and Organic Salts Based on Penicillin g and Amoxicillin Hydrolysate Derivatives against Resistant Bacteria. Pharmaceutics 2020, 12 (3), 221.
18. Fischer, C. L.; et al. Antibacterial Activity of Sphingoid Bases and Fatty Acids against Gram-Positive and Gram-Negative Bacteria. Antimicrobial Agents and Chemotherapy 2012, 56 (3), 1157-1161.
19. Florio, W.; et al. Comparative Evaluation of Antimicrobial Activity of Different Types of Ionic Liquids. Materials Science & Engineering C 2019, 104, 109907.
20. Garcia, M. T.; et al. Self-Assembly and Antimicrobial Activity of Long-Chain Amide-Functionalized Ionic Liquids in Aqueous Solution. Colloids and surfaces, B, Biointerfaces 2014, 123, 318-325.
21. Gundolf, T.; et al. Bacterial Resistance Toward Antimicrobial Ionic Liquids Mediated by Multidrug Efflux Pumps. Frontiers in Microbiology 2022, 1785.
22. Guo, J.; et al. Antibacterial Amino Acid-Based Poly(lonic Liquid) Membranes: Effects of Chirality, Chemical Bonding Type, and Application for MRSA Skin Infections. ACS Appl Bio Mater 2019, 2 (10), 4418-4426.
23. Hallett, J. P.; et al. Room-Temperature Ionic Liquids: Solvents for Synthesis and Catalysis. 2. Chemical Reviews 2011, 111 (5), 3508-3576.
24. Hou, X.-D.; et al. Evaluation of Toxicity and Biodegradability of Cholinium Amino Acids Ionic Liquids. PLoS One 2013, 8 (3), e59145.
25. Ibsen, K. N.; et al. Mechanism of Antibacterial Activity of Choline-Based Ionic Liquids (CAGE). ACS Biomater Sci Eng 2018, 4 (7), 2370-2379.
26. Islamipour, Z.; et al. Biodegradable Antibacterial and Antioxidant Nanocomposite Films Based on Dextrin for Bioactive Food Packaging. Journal of Nanostructure in Chemistry 2022.
27. Jeong, S.; et al. Elucidation of Molecular Interactions between Lipid Membranes and Ionic Liquids Using Model Cell Membranes. Soft Matter 2012, 8 (20), 5501-5506.
28. Kemp, T. J. Ionic Liquids—Pharmaceutical Potential. Science Progress 2012, 95 (2), 224.
29. Kiani, M.; et al. Multifunctional Green Synthesized Cu—Al Layered Double Hydroxide (LDH) Nanoparticles: Anti-Cancer and Antibacterial Activities. Scientific Reports 2022, 12 (1), 9461.
30. Kohanski, M. A.; et al. Sublethal Antibiotic Treatment Leads to Multidrug Resistance via Radical-Induced Mutagenesis. Molecular Cell 2010, 37 (3), 311-320.
31. Kubo, A.-L.; et al. Antimicrobial Activity of Polyoxometalate Ionic Liquids (POM-ILs) against Clinically Relevant Pathogens. Toxicol Lett 2017, 280, S193-S193.
32. Lee, J. Y.; et al. Effects of Ionic Liquid Alkyl Chain Length on Denaturation of Myoglobin by Anionic, Cationic, and Zwitterionic Detergents. Biomolecules 2019, 9 (7), 264.
33. Ma, H.; et al. Modeling Diversity in Structures of Bacterial Outer Membrane Lipids. J Chem Theory Comput 2017, 13 (2), 811-824.
34. Ma, H.; et al. Simulating Gram-Negative Bacterial Outer Membrane: A Coarse Grain Model. J Phys Chem B 2015, 119 (46), 14668-14682.
35. Mazraeh, M.; et al. Multifunctional Tetracycline-Loaded Silica-Coated Core-Shell Magnetic Nanoparticles: Antibacterial, Antibiofilm, and Cytotoxic Activities. ACS Applied Bio Materials 2022, 5 (4), 1731-1743.
36. Mester, P.; et al. Antimicrobial Effects of Short Chained Imidazolium-Based Ionic Liquids-Influence of Anion Chaotropicity. Ecotoxicology and Environmental Safety 2015, 111, 96-101.
37. Mester, P.; et al. FTIR Spectroscopy Suggests a Revised Mode of Action for the Cationic Side-Chain Effect of Ionic Liquids. ACS Combinatorial Science 2019, 21 (2), 90-97.
38. Mustahil, N. A.; et al. Synthesis, Characterization, Ecotoxicity and Biodegradability Evaluations of Novel Biocompatible Surface Active Lauroyl Sarcosinate Ionic Liquids. Chemosphere (Oxford) 2019, 229, 349-357.
39. Nikfarjam, N.; et al. Antimicrobial Ionic Liquid-Based Materials for Biomedical Applications (Adv. Funct. Mater. 42/2021). Adv Funct Mater 2021, 31 (42), 2170312-n/a.
40. Pendleton, J. N.; et al. The Antimicrobial Potential of Ionic Liquids: A Source of Chemical Diversity for Infection and Biofilm Control. Int J Antimicrob Agents 2015, 46 (2), 131-139.
41. Pernak, J.; et al. Anti-Microbial Activities of Ionic Liquids. Green Chem 2003, 5 (1), 52-56.
42. Pernak, J.; et al. Choline-Derivative-Based Ionic Liquids. Chemistry: a European journal 2007, 13 (24), 6817-6827.
43. Pernak, J.; et al. Synthesis and Anti-Microbial Activities of Choline-like Quaternary Ammonium Chlorides. Eur J Med Chem 2003, 38 (11), 1035-1042.
44. Petkovic, M.; et al. Novel Biocompatible Cholinium-Based Ionic Liquids—Toxicity and Biodegradability. Green Chem 2010, 12 (4), 643-64; 649.
45. Qin, J.; et al. Synthesis of Pyrrolidinium-Type Poly(lonic Liquid) Membranes for Antibacterial Applications. ACS Appl Mater Interfaces 2017, 9 (12), 10504-10511.
46. Rebelo, L. P. N.; et al. Accounting for the Unique, Doubly Dual Nature of Ionic Liquids from a Molecular Thermodynamic and Modeling Standpoint. Accounts of Chemical Research 2007, 40 (11), 1114-1121.
47. Saraswat, J.; et al. Synergistic Antimicrobial Activity of N-Methyl Substituted Pyrrolidinium-Based Ionic Liquids and Melittin against Gram-Positive and Gram-Negative Bacteria. Appl Microbiol Biotechnol 2020, 104 (24), 10465-10479.
48. Silhavy, T. J.; et al. The Bacterial Cell Envelope. Cold Spring Harb Perspect Biol 2010, 2 (5), a000414-a000414.
49. Singh, G.; et al. Antimicrobial Colloidal Complexes of Lysozyme with Bio-Based Surface Active Ionic Liquids in Aqueous Medium. J Phys Chem B 2020, 124 (18), 3791-3800.
50. Siopa, F.; et al. Choline-Based Ionic Liquids: Improvement of Antimicrobial Activity. ChemistrySelect (Weinheim) 2016, 1 (18), 5909-5916.
51. Tsuchido, T.; et al. Death Kinetics of Escherichia Coli in a Combined Treatment with Heat and Monolaurin [Antimicrobial Agents, Food Safety]. J Food Saf 1981, 3 (2), 57-68.
52. United States Centers for Disease Control and Prevention. Antibiotic Resistance Threats in the United States; 2019.
53. Wang, C.; et al. Bacteria-Activated Chlorin E6 Ionic Liquid Based on Cation and Anion Dual-Mode Antibacterial Action for Enhanced Photodynamic Efficacy. Biomaterials Science 2019, 7 (4), 1399-1410.
54. Wang, P.; et al. Limitations of MTT and MTS-Based Assays for Measurement of Antiproliferative Activity of Green Tea Polyphenols. PLoS One 2010, 5 (4), e10202.
55. Wasserscheid, P.; et al. Ionic Liquids in Synthesis: Second Edition; 2008; Vol. 1.
56. World Health Organization. Antimicrobial resistance; 2021.
57. Yang, D. D.; et al. Synergistic Interactions of Ionic Liquids and Antimicrobials Improve Drug Efficacy. iScience 2021, 24 (1), 101853.
58. Yazdani, A.; et al. Microbial Biocompatibility and Biodegradability of Choline-Amino Acid Based Ionic Liquids. Journal of Microbial & Biochemical Technology 2016, 8 (5), 415-421.
59. Zakrewsky, M.; et al. Choline and Geranate Deep Eutectic Solvent as a Broad-Spectrum Antiseptic Agent for Preventive and Therapeutic Applications. Adv Healthc Mater 2016, 5 (11), 1282-1289.
60. Zakrewsky, M.; et al. Ionic Liquids as a Class of Materials for Transdermal Delivery and Pathogen Neutralization. Proceedings of the National Academy of Sciences—PNAS 2014, 111 (37), 13313-13318.
61. Zheng, Z.; et al. Structure-Antibacterial Activity Relationships of Imidazolium-Type Ionic Liquid Monomers, Poly(lonic Liquids) and Poly(lonic Liquid) Membranes: Effect of Alkyl Chain Length and Cations. ACS Appl Mater Interfaces 2016, 8 (20), 12684-12692.

1. An ionic liquid comprising choline and an anion comprising the conjugate base of a substituted or unsubstituted C2-C20 linear or branched fatty acid.

2. The ionic liquid of claim 1, wherein the fatty acid comprises a saturated fatty acid, a monounsaturated fatty acid, a polyunsaturated fatty acid, or any combination thereof.

3. The ionic liquid of claim 1, wherein the anion comprises butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof.

4. The ionic liquid of claim 1, wherein a molar ratio of the choline to the anion is from about 1:1 to about 1:4.

5. (canceled)

6. The ionic liquid of claim 1, wherein the ionic liquid comprises choline decanoate having a molar ratio of choline to decanoic acid of about 1:2, choline 3-decenoate having a molar ratio of choline to 3-decenoic acid of about 1:2, choline 2-decenoate having a molar ratio of choline to 2-decenoic acid of about 1:2, or any combination thereof.

7. A composition comprising the ionic liquid of claim 1, wherein the composition is substantially free of alcohol.

8. A composition comprising the ionic liquid of claim 1, where the ionic liquid is used to modify the surface of another material.

9. The composition of claim 8, where the other material comprises a polymeric substrate.

10. The composition of claim 9, where the polymeric substrate comprises nanoparticles that are less than 1000 nm in size.

11. The composition of claim 7, further comprising at least one pharmaceutically-acceptable carrier or excipient.

12. (canceled)

13. The ionic liquid of claim 1, wherein the ionic liquid has a minimum bactericidal concentration (MBC) for E. coli of from about 0.5 mM to about 1750 mM.

14. (canceled)

15. The ionic liquid of claim 1, wherein the ionic liquid has a minimum bactericidal concentration (MBC) for methicillin-resistant Staphylococcus aureus (MRSA) of from about 0.5 mM to about 3 mM.

16. (canceled)

17. A method for treating or preventing an infection caused by bacteria in a subject, the method comprising administering the composition of claim 7 to the subject.

18. The method of claim 17, wherein the subject is a mammal or bird.

19. The method of claim 18, wherein the mammal is a human, cat, dog, horse, cattle, sheep, goat, hamster, guinea pig, rabbit, mouse, or rat.

20. The method of claim 18, wherein the bird is a chicken, turkey, duck, goose, or parrot.

21. The method of claim 17, wherein the ionic liquid or composition is administered topically, intranasally, or intravenously.

22.-23. (canceled)

24. The method of claim 17, wherein the bacteria comprise Gram-positive bacteria, Gram-negative bacteria, or both.

25. The method of claim 19, wherein the Gram-negative bacteria comprise E. coli.

26. The method of claim 19, wherein the Gram-positive bacteria comprise methicillin-resistant Staphylococcus aureus (MRSA) or Mycobacterium tuberculosis.

27. (canceled)
